(19) United States
(12) Patent Application Publication
(10) Pub. No.: US 2002/0016604 A1
(43) Pub. Date: Feb. 7, 2002

(54) Guidewire with serrated element

(76) Inventors: Robert Boock, Minnetonka, MN (US); Jeffrey A. Lee, Plymouth, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/872,773

(22) Filed: Jun. 1, 2001

Related U.S. Application Data
(63) Continuation-in-part of application No. 09/371,267, filed on Aug. 10, 1999, now Pat. No. 6,322,572.

(51) Int. Cl. A61B 17/22

(52) U.S. Cl. 606/159

(57) ABSTRACT

The present invention includes a guidewire with a distal end capable of boring into a thrombus. The guidewire comprises a serration element and may further comprise a distal end that includes a loop.

### CLAIM TO PRIORITY

This application is a continuation-in-part of U.S. patent application Ser. No. 09/371,267, filed Aug. 10, 1999.

### BACKGROUND OF THE INVENTION

The present invention relates to a guidewire with a serrated distal end and to a method for boring into a thrombus.

Brain attack afflicts more than 700,000 people in the United States annually. About 70 to 85% of brain attack episodes are attributable to ischemic stroke, which carries a mortality of 15-33%. Emerging treatments for acute cerebral ischemia include thrombolytic treatment. One type of thrombolytic treatment involves an early use of clot lysing agents and a subsequent restoration of blood flow. One lysing agent, rt-PA has been shown to be effective in restoring circulation and in reducing the overall morbidity. However, the benefits of rt-PA are effective only if treatment begins within the first 90 minutes to 3 hours after the initial ictus.

Currently, trials are underway to reduce the complications associated with this treatment by using local intra-arterial versus systemic intravenous delivery as well as the potential use of other low cost alternative thrombolytic agents. There have also been advances in imaging technologies such as perfusion MRI, CT angiography, and advances in diagnostic blood tests all geared to the early diagnosis of stroke to speed the treatment and expand the efficacy of these early interventions.

Other types of stroke treatment include early imaging, and a creation of dedicated stroke centers. All of these treatments have brought greater emphasis to the early treatment of stroke. A key to this treatment is a re-establishment of blood flow as early as possible to limit ischemic brain damage. The difficulty with thrombolysis alone is that this technique depends upon several variables, clot type, clot density, location, metabolism and so forth which adversely impact the effectiveness of this treatment.

Several devices have been designed for peripheral clot disruption. One device is described in U.S. Pat. No. 5,779,721 ('721), which issued Jul. 14, 1998. The '721 patent describes a system for opening a lumen in an occluded blood vessel. The system includes a working head for revascularizing the blood vessel and a mechanism for extracting or removing debris produced by operation of the working head. The working head is a rotary impacting impeller. The mechanism for extracting or removing debris introduces an infusate liquid into the blood vessel adjacent the working head and withdraws the liquid and some blood from the vessel. The infusate liquid may include a lytic drug such as heparin or urokinase. The blood and infusate liquid are remotely collected.

Thrombectomy devices may be utilized as a part of the system. One device is the Amplatz Thrombectomy Device designated by the trademark CLOT BUSTER by Microvena Corporation. Another device is the Craig thrombectomy Brush.

### SUMMARY OF THE INVENTION

One embodiment of the present invention includes a guidewire comprising a serrated distal end.

Another embodiment of the present invention includes a guidewire comprising a wire portion and at least one serrated portion attached to the wire portion.

One other embodiment of the present invention includes a method for boring or channeling into and/or through a thrombus. The method includes providing a guidewire with a serration element and transporting the serration element to a thrombus. The guidewire is used to bore the serration element into the thrombus. The guidewire is also used to tunnel or channel through the thrombus, as well.

### DESCRIPTION OF THE DRAWINGS

FIG. 1 is a transverse cross-sectional view of one embodiment of the serrated guidewire assembly of the present invention.

FIG. 2 is a side view of one embodiment of the serrated sleeve element of the guidewire of the present invention.

FIG. 3 is a side view of one embodiment comprising a series of serrated, scooped sleeves of the guidewire of the present invention.

FIG. 4 is a side view of one looped embodiment of the guidewire of the present invention.

FIG. 5 is a transverse, cross-sectional view of one embodiment of a serration configuration of the serrated element of the guidewire of the present invention.

FIG. 6 is a transverse, cross-sectional view of another embodiment of a serration configuration of the guidewire of the present invention.

FIG. 7 is transverse cross-sectional view of one woven, serrated embodiment of a serration configuration of the guidewire of the present invention.

FIG. 8A is a side view of a double-looped embodiment of the guidewire of the present invention.

FIG. 8B is a side view of a hooked or shaped, i.e. j- or shallow curve shaped, embodiment of the guidewire of the present invention.

FIG. 8C is a side view of one other looped embodiment of the guidewire of the present invention.

FIG. 8D is a side view of a partial loop embodiment of the guidewire of the present invention.

FIG. 9 is a schematic view of one guidewire embodiment boring through a thrombus.

FIG. 10 is a side view of one other guidewire with discrete “scoops” embodiment of the present invention.

FIG. 11 is a side view of one embodiment of the serrated guidewire with a wire loop basket surrounding the serrated portion. The basket is used to center the serrated portion with the vessel.

FIG. 12 is a side view of an embodiment of the serrated guidewire with baskets on both sides of the serrated portion to center the device with the vessel. Multiple baskets and a serrated sections could be used.

FIG. 13A is a cross-sectional view of one embodiment undeployed with a standard microcatheter.

FIG. 13B is the microcatheter of 13A in a deployed position in which a continuous basket created by pre-shaping a memory material into a basket in which the serrated portion remains with the center of the basket.

### DETAILED DESCRIPTION OF EMBODIMENTS

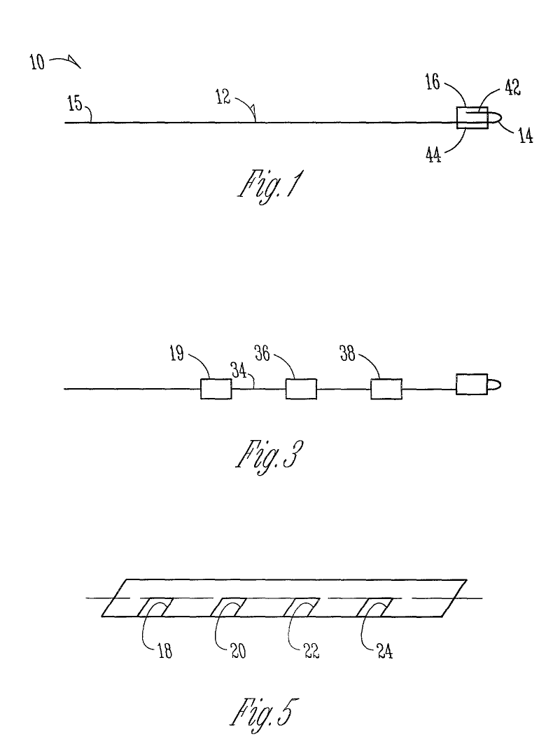

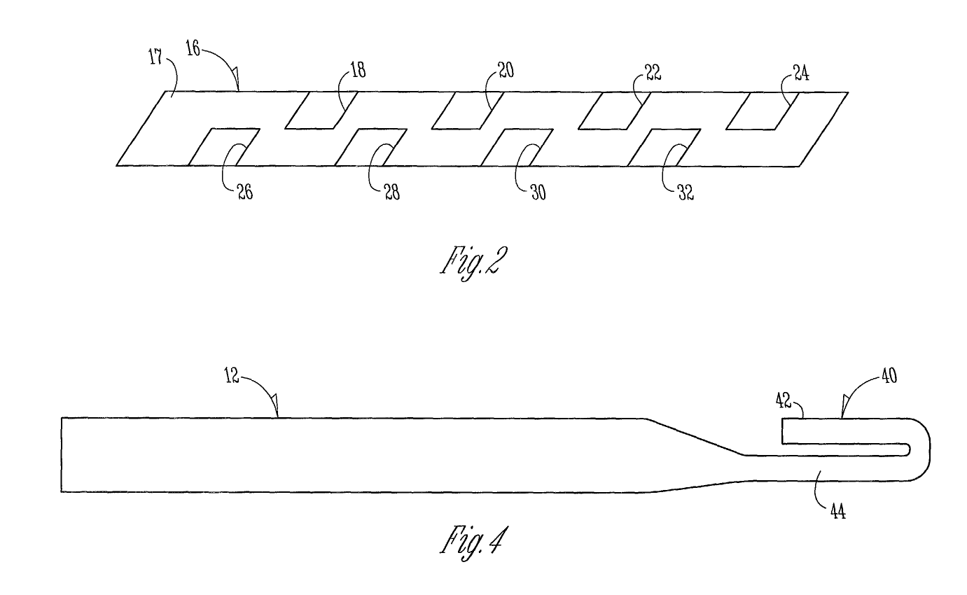

One embodiment of the serrating guidewire of the present invention, illustrated generally at 10 in FIG. 1, includes a wire portion 12 which has a proximal portion 15 and a distal end 14 and at least one serrated element 16 which is at or proximal to the distal end 14. In one embodiment, the serrated element 16 comprises a hollow, sleeve configured main body 17 that includes skived regions 18, 20, 22, 24, 26, 28, 30 and 32, shown in FIG. 2. In another embodiment, the serrated element 16 is comprised of discrete scoops 34, 36, and 38, as shown in FIG. 3.

The serrated element 16 may be attached to the wire portion 12 by bonding to the wire portion 12 by fabrication techniques that include gluing or heat shrinking reflowing base material. In another embodiment, the wire portion 12 is looped as shown at 40 in FIG. 4. Two ends of the loop 42 and 44 are captured within the serrated element 16 as shown in FIG. 1. Tension from the two ends 42 and 44 aids in retaining the serrated element 16 in a hollow open conformation.

The wire portion 12 is made of a biocompatible material such as stainless steel wire or Nitinol wire. The serrated element 16 is made of a material such as TFE shrink tubing, polyimide tubing, polyethylene tubing or high density polyethylene tubing. One benefit of using a polymeric material, especially a somewhat “soft” polymeric material, is that the serrated element 16 will not damage a vessel wall as the guidewire with the serrated element 10 is transported to a thrombus site. However, the serrated element 16 can be fabricated to have sharp edges at the serrations even though the surface is soft. Thus, the serrated element has features that render it transportable within a blood vessel while retaining its effectiveness in boring through a thrombus.

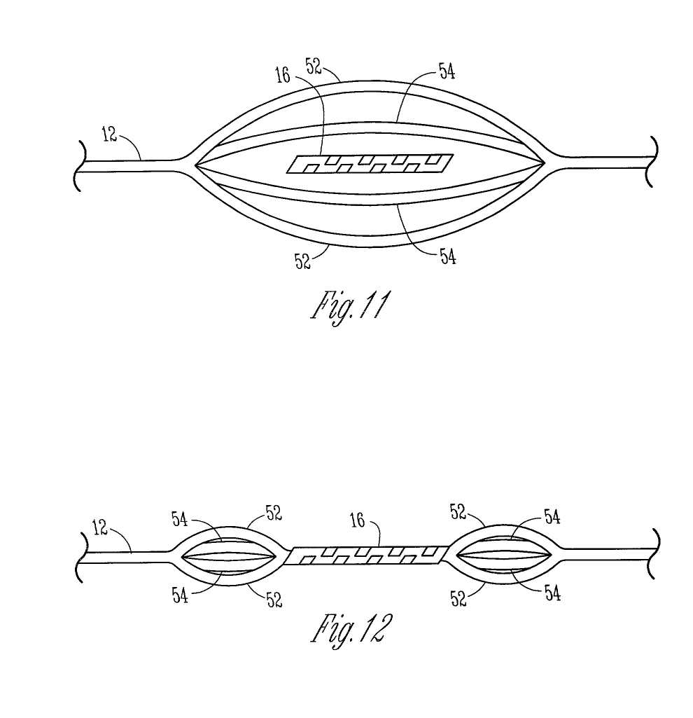

When the serrated element 16 of the guidewire 10 is positioned within a thrombus, serrations 18, 20, 22, 24, and 26, shown in FIG. 2, or scoops 34, 36 and 38 shown in FIG. 3, defined by the serrated element 16 bore through the thrombus and create a channel through the thrombus in order to re-establish blood flow and to create more surface area to enhance lytic attack on the clot as shown in FIG. 9. Because the serrated element 16 is hollow, the element 16 acts to core the channel and to allow drug access into the thrombus.

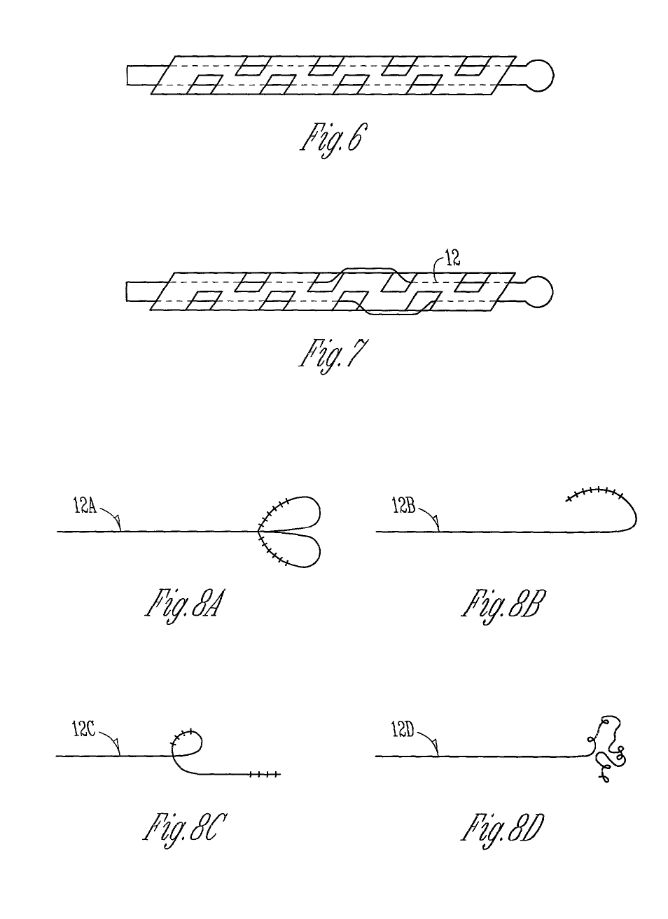

The serrated element 16 may be skived to create slots of about 2 mm in length that are spaced about 2 to 4 mm apart, although it is appreciated that dimensions may be adjusted to perform particular functions. The serrations 18-24 may be cut on one side of the element as is shown in FIG. 5 or may be cut on alternating sides of the main body 17 or in a spiral pattern around the main body as shown in FIG. 6. In another embodiment, shown in FIG. 7, the wire portion 12 is woven through one or more serrations. In another embodiment shown at 50 in FIG. 10, a series of serrated elements 16A, 16B, 16C and 16D are bonded to the wire portion 12. The most distal element 16D may enclose ends of the loop 40.

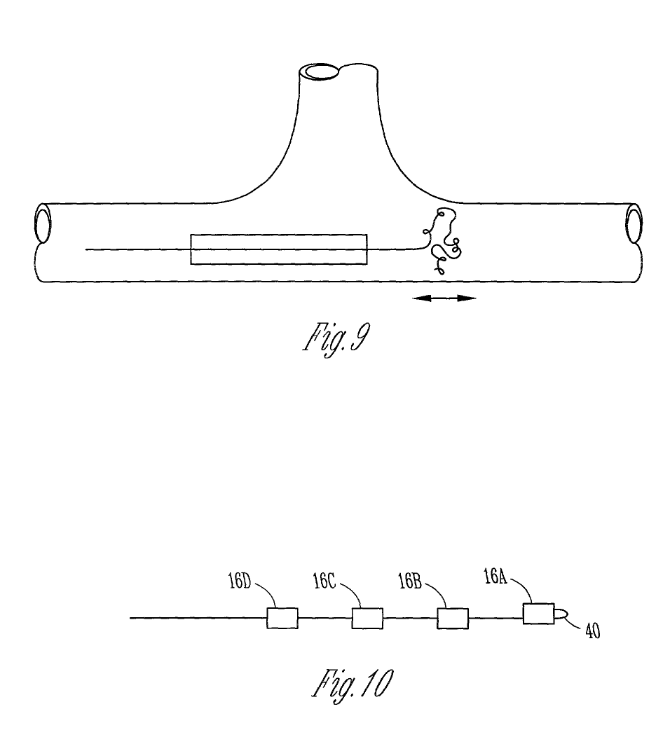

For looped end embodiments, one of which is shown in FIG. 4, where the wire portion 12 is made of a memory material such as Nitinol, the loop may be imparted with a variety of configurations. Some of these configurations are shown in FIGS. 8A, 8B, 8C and 8D. These wire configurations may also include discrete or continuous serrations fabricated into the wire portion 12A-12D of the guidewire. The looped configurations aid the serration element 16 in boring through a thrombus by providing local maceration.

A metal may be employed for the wire portion 12 because metal typically has the requisite strength and resistance to deformation which are necessary for the guidewire's distal end to traverse a blood vessel. While a metal is described, it is contemplated that polymeric-based materials capable of retaining a memory-imparted conformation and having the requisite strength and stiffness are suitable for use in the distal guidewire end of the present invention.

It is further contemplated that the biocompatible material and biocompatible, memory Nitinol wire or polymeric material may be coated. In one embodiment, the wire 12 and/or the serrated sleeve element 16, may be coated with a coating that comprises or absorbs a lytic drug. With this embodiment, the lytic drug is delivered in intimate contact with the particle of thrombus captured within the serrations 18, 20, 22, 24, and 26. In another embodiment, the wire portion 12 and/or sleeve 16 is coated with an abrasive coating. The abrasive coating further aids in breaking up and dispersing a thrombus. The wire portion 12 and sleeve 16 may be coated with a hydrophilic coating or with a coating array fabricated for orchestrated drug delivery at a thrombus site.

The guidewire of the present invention may be transported to a treatment site with virtually any conventional catheter or microcatheter usable with a guidewire. For guidewire embodiments that employ distal loops such as are shown in FIGS. 8A-8D, the memory-imparted or compressed loops may be expanded when the distal end 14 of the guidewire 10 is pushed out of the microcatheter.

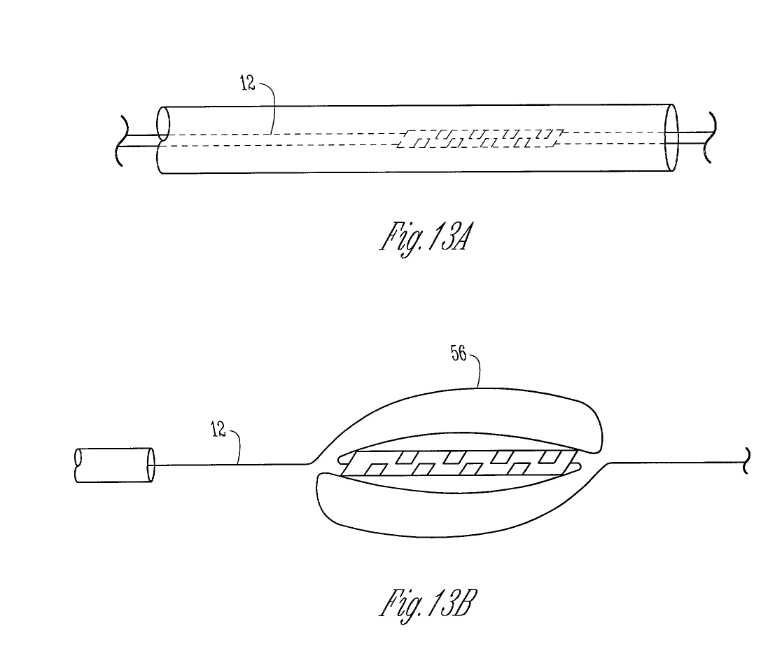

For basket embodiments, which are shown in FIGS. 11, 12, and 13, a preformed basket comprised of wire loops shown at 52, 54 either surrounds the serrated portion 16 or lies at either end of the serrated portion 16, 52 and 54 in FIG. 12. Another embodiment has a continuous shape-memory basket shown at 56 in FIG. 13B. Upon deployment, this embodiment takes the pre-formed basket shape surrounding the skived/serrated portion 16. The basket aids in centering the serrated portion to open a central channel and eliminate any vessel wall contact with the serrated portions.

The guidewire of the present invention may be used to treat stroke (brain attack) and other conditions caused by thrombus formation. It is believed that the guidewire of the present invention permits a physician to combine thrombus boring with localized drug delivery to rapidly mitigate effects of thrombus formation.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described and detailed herein. Rather, reference should be made to the appended claims as indicative of the scope and content of the present invention.

What is claimed is:

1. A guidewire comprising a serrated distal end.

2. A guidewire comprising a wire portion and at least one serrated portion attached to the wire portion.

3. The guidewire of claim 1 wherein serrations of the serrated distal end are sized to tear, channel or bore through a thrombus.

4. The guidewire of claim 2 wherein the serrated portion is hollow.

5. The guidewire of claim 2 wherein the serrated portion has a soft surface.

6. The guidewire of claim 2 wherein the serrated portion is joined to the wire portion.

7. The guidewire of claim 2 wherein the wire portion terminates in a looped distal end.

8. The guidewire of claim 7 wherein the serrated portion encloses opposing ends of the looped distal end.

9. The guidewire of claim 2 wherein the wire portion terminates in a hooked, j, or shallow curved distal end.

10. The guidewire of claim 2 wherein the wire portion terminates in a double loop.

11. The guidewire of claim 2 and further including a coating for coating either the wire portion or the serrated element or both the wire portion and the serrated element.

12. The guidewire of claim 11 wherein the coating comprises a lytic material.

13. A method for boring into a thrombus, comprising:
providing a guidewire with a serrated element;
transporting the guidewire and serrated element to a thrombus; and
pushing the serrated element into the thrombus so that the serrated element bores into the thrombus.

14. The method of claim 13 and further including delivering lytic drugs proximal to or into the thrombus.

15. A guidewire comprising a serrated portion and a basket positioned about the serrated portion.

16. A guidewire comprising a serrated portion and at least one basket positioned proximal to the serrated portion.

17. The guidewire of claim 16 and further comprising a second basket positioned proximal to the serrated portion.

18. The guidewire of claim 15 wherein the basket has a collapsed position wherein the basket is collapsed about the serrated portion and an expanded position wherein the basket is expanded over the serrated region.

19 The guidewire of claim 15 and further comprising a catheter wherein the guidewire is positioned within the catheter.
